(19) United States
(12) Patent Application Publication
(10) Pub. No.: US 2010/0069254 A1
(43) Pub. Date: Mar. 18, 2010

(54) Cell Culture Model for Demyelination/Remyelination

(76) Inventor: Laree Hiser, Brandon, MS (US)

Correspondence Address: STITES & HARBISON PLLC, 401 COMMERCE STREET, SUITE 800, NASHVILLE, TN, 37219, US

(21) Appl. No.: 12/211,685

(22) Filed: Sep. 16, 2008

(51) Int. Cl. C40B 30/04; C12Q 1/02; C12N 5/06; C12Q 1/68

(52) U.S. Cl. 506/9; 435/29; 435/6; 435/325

(57) ABSTRACT

A research model for monitoring demyelination or remyelination in a sample of cells that comprises providing cells, typically CNS cells, and contacting cells with a demyelination solution such as one that includes one of hexachlorophene and/or lysophosphatidylcholine.

### PRIORITY INFORMATION

This application claims benefit to U.S. Application No. 60/624,738, filed Nov. 2, 2004, the contents of which are incorporated herein by reference.

### FIELD OF THE INVENTION

The present invention relates to the field treating multiple sclerosis (MS). Additionally, the present invention relates to developing a cell culture model that is amenable to studying the effects of demyelination on the neurons and glial cells. Additionally, the present invention relates to a process of demyelination of CNS axons. Additionally, the present invention relates to the field of developing molecular technologies that protect neurological tissues during cranial radiation therapy (CRT) for brain tumors. Further embodiments of the present invention relate to the fields of spinal cord injury study and repair, as well as diseases and disorders involving myelination. Additionally, the present invention relates to the field of studying the molecular aspects of remyelination. Additionally, the present invention relates to the field of drug screening and testing for therapies aimed at preventing demyelination, enhancing remyelination or stabilizing demyelinated neurons.

### BACKGROUND OF THE INVENTION

Multiple sclerosis (MS) is a chronic, disabling disease that affects approximately 400,000 Americans and 2.5 million people worldwide. Although the symptoms seem to result from demyelinaton of axons in the central nervous system, the biochemical mechanisms are poorly understood. A better understanding of these mechanisms could lead to therapies for symptom management or even prevention. The present invention assists in developing such an understanding.

The growth of myelinated axons in culture is ideal for biochemical and genomic studies because of the virtually unlimited availability of material and the ability to perform carefully controlled experiments. Differentiation of mouse embryonic stem cells with retinoic acid produces myelinated axons in culture (Liu, et al., 2000). We have modified this protocol to establish cultures exhibiting compact myelin of high quality as demonstrated by transmission electron microscopy (FIG. 1).

Brain tumors are the second most common form of cancer in children, accounting for 21% of all pediatric cancers. Cranial radiation therapy (CRT) has been the standard treatment of pediatric brain tumors for several decades, often in combination with neurosurgery and chemotherapy. Survival rates for children with brain tumors have increased over the past three decades with five-year survival rates now ranging between 60% and 80% following the use of radiation therapy (Stewart & Cohen, 1998).

The increasing number of survivors of childhood brain tumors has uncovered deleterious late effects as a result of CRT. These include declines in intelligence, memory, language, attention, academic skills, psychosocial function, and quality of life and have been observed six months to several years postirradiation (Dennis, et al., 1996). Older children fare better than those who are younger at the time of treatment (Mulhem, et al., 1998). This suggests that the negative effects of CRT are more severe in children than in adults because brain development is incomplete.

Radiation-induced changes in the central nervous system (CNS) are responsible for tumor destruction but also have detrimental acute and long-term neurocognitive effects. The neuorcognitive deficits have been shown to be associated with observable changes in white matter in pediatric patients (Mulhem et al., 1999) and are attributed, in part, to destruction of myelin (Schultheiss et al., 1995). Myelin loss occurs shortly after radiation treatment and persists for several years. Axonal loss is observed as a late effect that is probably secondary to demyelination (Virta, et al., 2000). Thus, protection of myelin during radiation therapy or enhancement of remyelination after treatment might alleviate the late effects and improve the quality of life for pediatric cancer survivors.

Prevention of demyelination during CRT may be difficult because in vitro experiments suggest that radiation triggers apoptosis of differentiated oligodendrocytes, the cells that form CNS myelin (Vrdoljak et al., 1992). However, the progenitor cells are spared, suggesting that remyelination is possible, especially considering that white matter continues to develop in children.

Demyelination has been shown to have profound effects on the axonal cytoskeleton (Zhu, et al., 1999). This is detrimental to the neuron because axonal transport is disrupted and the diameter of the axon is reduced, which affects nerve conduction. Other neuronal changes in response to demyelination include redistribution of ion channels along the axon (Waxman, et al., 1995). Without healthy neurons, repair of myelin is less likely to occur. Without being bound by theory or mechanism, sustaining the signals normally found between myelin and axons during radiation treatment will stabilize the neurons and allow remyelination to occur more efficiently.

Little is known regarding the normal signaling pathways or how they are perturbed as a result of demyelination. One receptor that has received recent attention is Nogo-66 (Liu, et al., 2002), but the downstream signaling pathway has not yet been identified. The present invention will allow for the identification of candidate genes that may be involved in signal transduction pathways. Future investigations can then be directed toward identifying good drug targets.

### SUMMARY OF THE INVENTION

One aspect of the present invention helps define the molecular mechanisms for maintaining stable microtubules within neurons and to find ways of preserving healthy microtubules, thus preventing disability in disease states such as multiple sclerosis. Secondary progressive MS responds poorly to current therapies and has been proposed to result from axonal transport deficits that are indirectly caused by demyelination. An understanding of the signaling processes between myelin and axonal microtubules is critical for developing interventions to reduce disability.

An additional aspect of the present invention is to identify genes that are up- or down-regulated in response to demyelination. In other embodiments of the present invention, the function of these gene products is determined. For example, the mechanism of stabilizing microtubules within CNS neurons is studied.

Microarrays are used to investigate mRNA levels on a genome-wide scale. The microgram quantities of RNA needed for these studies are obtained from CNS-like cell cultures derived from mouse embryonic stem cells. Reference and experimental RNA samples are extracted from cultures that differ only in their state of myelination. As an example, to produce demyelinated samples, cultures are treated with about 10 μg/ml hexachlorophene for about 10 minutes.

Software packages specifically designed for microarray analysis, such as ArrayInformatics (Perkin Elmer) and GeneSpring (Agilent Technologies), are used for normalization of data and cluster analysis of multiple data sets. Reproducible two-fold differences in gene expression are considered significant.

The methods of the present invention can assist in the understanding of the interactions between myelin and axons that in turn can lead to the design of interventions to prevent demyelination, stabilize the health and function of demyelinated neurons, and/or enhance remyelination.

### BRIEF DESCRIPTION OF THE DRAWINGS AND ATTACHMENTS

The following drawings are given for illustration of embodiments of the present invention, and are not intended to be limiting thereof.

FIG. 1 is transmission electron micrographs of cell cultures. Panel A shows a myelinated axon from a control culture. Panel B is from a culture treated for 10 minutes with 10 μg/ml hexachlorophene and then immediately fixed for electron microscopy.

FIG. 2 shows anti-galactocerebroside immunofluorescence micrographs of differentiated ES-D3 cells grown in media that A) was not permissive for myelination of axons or B) allowed formation of myelin.

FIG. 3 shows metabolic assays of demyelinated cell cultures. Cultures were treated for 10 minutes with hexachlorophene (panel A) or lysophosphatidylcholine (panel B). Metabolic activity of cells was then measured by an MTS assay as described in the text.

FIG. 4 is a flow chart showing an embodiment of the present invention, namely showing an example of a cell culture model for myelination/demyelination.

### DESCRIPTION OF THE INVENTION

Clinical problems associated with a number of disorders such as multiple sclerosis and spinal cord injury are believed to be the result of demyelination of CNS axons. Embodiments of the present invention lead the way to the identification of molecular targets for drug therapies that will decrease rates of demyelination and/or enhance remyelination. Additionally, the cell culture model provides a convenient and cost-effective method for preliminary testing of drug therapies.

The study of complex central nervous system (CNS) diseases such as multiple sclerosis (MS) has been hindered by the lack of research models. Because several cell types, most notably those of the immune and nervous systems, are involved, it is difficult to dissect the biochemical pathways that are responsible for any one aspect of the disease. The present inventor has developed a cell culture model of demyelination that reduces the complexity of the problem by eliminating lymphocytes. Also, the present invention eliminates the need for human and animal subjects.

Thus, gene expression that is directly affected by demyelination without complicating effects such as exposure to cytokines can be determined. This should permit the identification of genes that are regulated in neurons and glial cells in response to demyelination.

Although the etiology of the disease process is still debated, it is generally accepted that the clinical effects of MS are due to demyelination of CNS axons. Oligodendrocytes are believed to be the primary target of an autoimmune response (Arredondo, Lovett-Racke and Racke, 2003), but the progression of disability is likely to be a result of disruption of neuronal function (Herndon, 2002). Thus, interventions aimed at stabilizing demyelinated axons could potentially reduce the clinical manifestations of the disease.

Demyelination is known to result in changes to axonal microtubules, but the mechanism for this intercellular event is unknown (Zhu, et al., 1999). One hypothesis is that there is a signaling pathway between oligodendrocytes and neurons that is interrupted upon removal of myelin. The absence of proper signaling could then lead to disruption of microtubules and impaired axonal transport. This would have long-term effects on neuronal health that would exacerbate the initial deficits in saltatory conduction resulting from demyelination.

It has been demonstrated that myelin impacts axonal health. In particular, proteolipid protein mutations have been shown to impair fast retrograde and anterograde axonal transport in a mouse model (Edgar, et al., 2004) and to cause neuroaxonal injury in humans (Bonavita, et al., 2001). Because proteolipid protein is the major CNS myelin protein, these studies suggest that demyelination such as occurs in MS is responsible for the axonal degeneration that leads to long-term disability.

Patients with chronic disabilities resulting from demyelinating diseases and disorders require extensive nursing care. Reduction of these disabilities would reduce the strain on health care systems with nursing shortages.

Changes in intracellular calcium levels have been associated with several neuronal pathologies, including Alzheimer's and Parkinson's diseases (Paschen and Frandsen, 2001). Intracellular calcium levels affect numerous biological processes and the mechanism for calcium-related injuries is not well understood. However, it should be noted that microtubules are calcium sensitive. High calcium concentrations cause depolymerization of microtubules in vitro. Disruption of microtubules has been observed in vivo following experimental axotomy, which induces elevation of intracellular calcium concentrations (Spira, Oren, Dormann, Ilouz, & Lev, 2001).

Destabilization of microtubules can lead to degeneration of neurons as a result of disruptions in axonal transport and a consequent shortage of structural proteins required for maintenance at the ends of axons (McQuarrie, Brady, & Lasek, 1986). Remaining neurons may sprout, a process that is associated with post-polio syndrome (Dalakas, 1995). Thus, the identification of signaling pathways that influence microtubule stability may lead to significant clinical outcomes.

The present invention is significant in allowing for the identification of genes which are regulated in response to demyelination of CNS-like axons and for evaluating the signaling mechanisms that affect microtubules in response to demyelination.

Microarray technology may be used to investigate changes in microtubule organization within axons that are mediated by the regulation of gene transcription as a result of demyelinating processes. RNA samples are collected from CNS-like neuronal cell cultures derived from mouse embryonic stem cells. Control RNA samples are taken from cultures of myelinated axons. Experimental samples are from cultures that differ from the control samples because they have been demyelinated.

Because demyelination may persist for years in MS patients, examination of the timing for gene expression is important. Time course experiments determine which genes are up- or down-regulated immediately following demyelination and which ones are differentially expressed at various times, including any range from a few minutes to several days after demyelination. In certain embodiments, the range may be from 24 to 96 hours. This helps identify genes that play important roles in chronic demyelination.

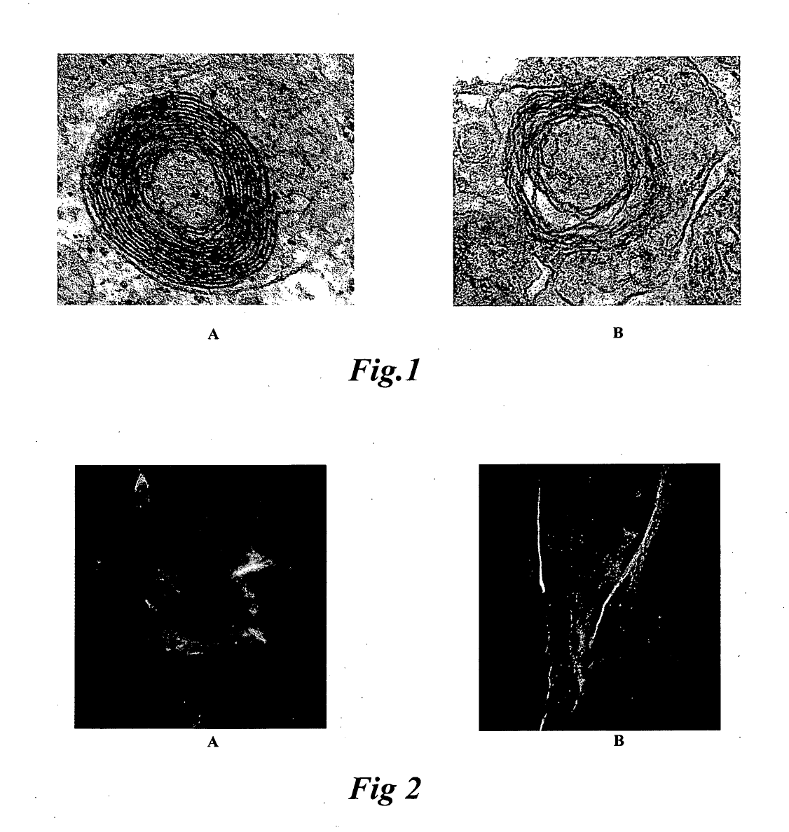

With embodiments of the present invention, mouse embryonic stem cells can be differentiated into CNS-like neurons and glial cells (Liu, et al., 2000). The growth medium and cell attachment substrates have been optimized to obtain cultures that form compact myelin within 10 to 15 days after differentiation. See FIG. 1A). Briefly, ES-D3 cells are maintained in the undifferentiated state using leukemia inhibitory factor to avoid contamination of cultures with feeder cells. In embodiments of the present invention, the differentiation process can require eight days, with exposure to retinoic acid for the last four days. The resulting embryoid bodies are dissociated and plated on dishes containing Matrigel. The differentiated cells are cultured in conditioned media (a mixture of NEUROBASAL™ supplemented with B27 and Dulbecco's modified Eagle medium containing serum) that permits growth of both neurons and glial cells.

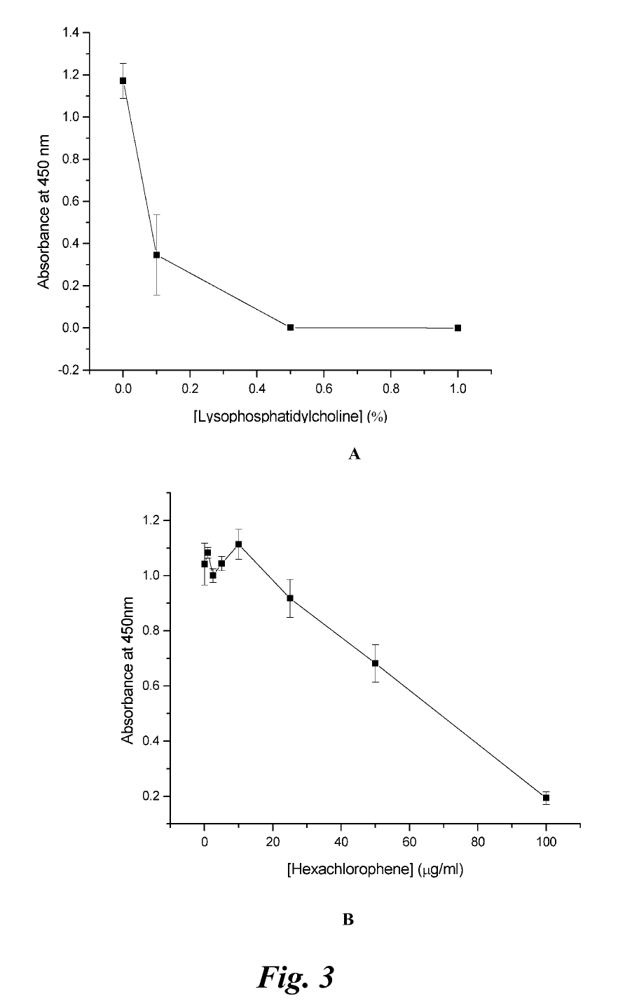

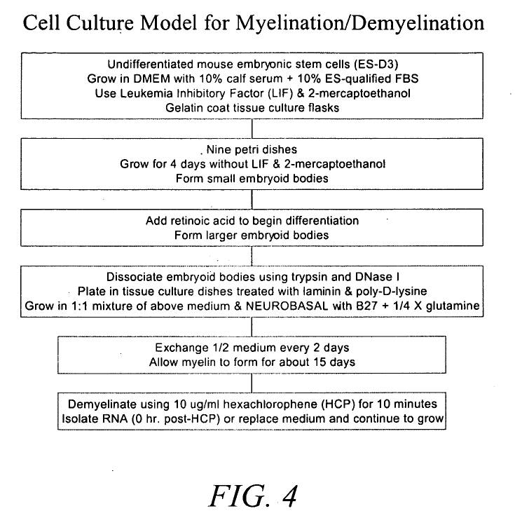

In the development of an in vitro model for demyelination, embodiments of the present invention include the use of chemicals (such as, for example, hexachlorophene and lysophosphatidylcholine) to achieve demyelination in culture. Monitoring of metabolic activity (CellTiter 96® AQueous One; Promega) is used to identify concentrations that caused a minimum amount of damage to the oligodendrocytes and neurons. In this assay a tetrazolium compound (MTS) and an electron coupling reagent (phenazine ethosulfate) are used for calorimetric detection of metabolic activity. MTS is bioreduced, presumably by NADPH or NADH, into a colored formazan product that is soluble in tissue culture medium and can be detected spectrophotometrically. Absorbance at about 450 nm is proportional to metabolic activity and is commonly used to monitor the number of living cells in culture. A 10 minute exposure to 10 μg/ml hexachlorophene is typically sufficient to cause significant demyelination (see FIG. 1B) without an observable effect on cellular metabolism (FIG. 3A). In contrast, even relatively low concentrations (0.1%) of lysophosphatidylcho line appeared to cause significant cellular damage (FIG. 3B). No significant spontaneous remyelination was observed by transmission electron microscopy up to two weeks after growth in normal culture medium after demyelination by hexachlorophene. As stated herein, the models of the present invention can be used to screen for compounds that promote remyelination.

The following examples show embodiments of the present invention and are not intended to be limiting thereof.

### EXAMPLE 1

Cell Culture.

Mouse embryonic stem cells (ES-D3) obtained from American Type Culture Collection are maintained in the undifferentiated state by growth on gelatin-coated flasks in the presence of leukemia inhibitory factor and 2-mercaptoethanol at 37° C. in a 5% CO₂ humid environment (Williams, et al., 1988). Cultures are restarted from frozen stocks every month to maintain the undifferentiated state. Culture medium is Dulbecco's Modified Eagle Medium (Invitrogen) with 10% embryonic stem cell qualified fetal bovine serum and 10% calf serum.

Differentiation into mixed cultures of neurons and CNS-type glial cells are by the 4−/4+ protocol described by Bain, Ray, Yao, and Gottlieb (1996). This involves growth on a substrate that is not permissive for cell attachment for four days during which time embryoid bodies form. Induction of differentiation is by addition of retinoic acid for an additional four days. The embryoid bodies are then dispersed and plated in the absence of retinoic acid on a substrate that permits cell attachment. The inventor has found that growth in a 1:1 mixture of the medium described above and Neurobasal™ medium (Invitrogen) supplemented with B27 (Invitrogen) is permissive for myelin formation. Tissue culture dishes and glass coverslips are coated with poly-D-lysine and laminin to enhance attachment of neurons (Krauss and Fischbach, 1998). For transmission electron microscopy, differentiated cells are grown on Matrigel™ cell culture inserts (BD Biosciences) and prepared for microscopy as described in Hutchins (1995). The formation of myelin is also monitored by immunofluorescence microscopy.

Demyelination of Cultures.

Several approaches have been used to successfully demyelinate neurons in animals. Examples within the scope of the present invention include ethidium bromide, lysophosphatidyl choline, hexachlorophene, as well as immunological methods. Additional methods include radiation (to mimic CRT) and the use of antibodies to myelin components followed by complement activation.

Hexachlorophene has been used at 0.1 to 0.2 μg/ml in tadpole bins to cause demyelination in Xenopus tadpoles (Reier, et al., 1978). Lysophosphatidyl choline is commonly injected into animals in the amount of 2 μl of a 1% solution to cause local demyelination. Concentrations that cause demyelination in cell cultures without significant damage to neurons or oligodendrocyte cell bodies is determined empirically using metabolic assays. Immunofluorescence microscopy using antibodies directed against both oligodendrocytes (e.g. anti-galactocerebroside) and neurons (e.g. anti-βIII tubulin) are used to assess the extent of demyelination. The degree of demyelination is confirmed by transmission electron microscopy.

Exposure to about 10 μg/ml hexachlorophene for about 10 min is sufficient for demyelination of neuronal cell cultures as demonstrated by transmission electron microscopy. This treatment is mild enough to prevent significant damage to the neural or glial cell bodies as measured by metabolic assays. Thus, hexachlorophene treatment is used for demyelination of neuronal cultures (15 days post-differentiation to allow good myelin formation) prior to RNA extraction. Immunological treatments such as complement activation and other chemicals such as lysophosphatidyl choline are alternative methods that may be used for demyelination to confirm the results obtained using hexachlorophene.

### EXAMPLE 2

Genomic Analysis.

Comparative hybridization of neuronal cultures with and without myelin is used to detect changes in gene expression that occur in response to demyelination. Total RNA samples are extracted from myelinated or demyelinated cell cultures differentiated from mouse embryonic stem cells in parallel. Total RNA is extracted using a Micro-to-Midi™ RNA Extraction kit or a TRIZOL reagent (both from Invitrogen), and contaminating genomic DNA is digested with deoxyribonuclease I amplification grade (Sigma). The quality of each RNA preparation is checked using RNA 6000 LabChips™ (Agilent) to assess degradation, and the RNA concentration is determined spectrophotometrically (1 OD at 260 nm=40 μg/ml). Approximately 10 μg of total RNA is needed for each experiment. This amount can be obtained from 1×10⁶ cells and is not a limitation in the cell cultures. Probes for the demyelinated and control cultures are generated by reverse transcription of total cellular RNA using Reflectase™ reverse transcriptase (Active Motif) and oligo(dT) primers. To maximize signal intensity, probes are labeled indirectly. Amino allyl dUTP is incorporated during the reverse transcription reaction. In a second step, dyes such as monofunctional forms of Cyanine 3 or Cyanine 5 are used to label the amine-modified cDNA. Unincorporated fluors are removed using a PCR purification kit (Qiagen), and RNA is hydrolyzed by addition of NaOH and incubation at 65° C. An alternative method for probe labeling is to directly incorporate fluors coupled to a nucleotide such as dCTP into the cDNA during the reverse transcription reaction. Because the bulky fluors cause inefficient incorporation of the modified nucleotide, direct incorporation may result in lower specific activity compared to the indirect labeling method.

The labeled probes are mixed in equal amounts and hybridized to microarrays containing mouse genes. Microarrays containing 15,264 sequence verified mouse expressed sequence tags are currently available from Microarray Centre. As the mouse genome sequence nears completion other microarrays representing more genes may become available. Hybridization and washing steps are conducted as recommended by the supplier using calf thymus DNA and yeast tRNA as blocking agents. Arabadopsis genes are used as positive controls.

The number of replicates required to achieve a predetermined level of statistical significance using power analysis is almost impossible to determine when using microarrays because so many genes are being analyzed and the variance and magnitude parameters are different for each gene (Yang & Speed, 2003). In general, eight replicate slides are sufficient for obtaining significance for 1.6-fold differences for the majority of the genes (Yang & Speed). These statistics are improved by careful experimental design to increase the degree of independence. Independent controls for each experiment are used rather than one reference RNA sample to decrease the number of microarrays needed to reach statistical significance for two-fold changes in the level of mRNA for any given gene.

Four types of replication are employed to address various statistical issues. Duplicate spots on each microarray provide quality information about the hybridization and the printing of the slide. Dye-swap, or reciprocally labeled, replicates are used to minimize systematic bias due to differences in the fluors used for labeling the control and experimental RNA samples. Technical duplicates—replicate slides hybridized with the same RNA preparations—reduce the variability due to hybridization and probe labeling. Biological triplicates using RNA extracted from independently differentiated cultures ensure the highest degree of independence feasible. The number of biological replicates are increased, as necessary, in order to identify genes that show increases or decreases of two-fold upon demyelination.

Data Analysis

Data are collected using ScanArray Express (Perkin Elmer). Software packages such as ArrayInformatics and QuantArray (Perkin Elmer) are used for normalization and data analysis, including cluster analysis. Reproducible two-fold differences in gene expression are considered significant.

Genes that are involved in signal transduction pathways between oligodendrocytes and the axonal cytoskeleton are anticipated to show differential expression. Thus, identified genes that are known to participate in signal transduction pathways are examined further. Other differentially expressed genes may also be of interest such as those involved in apoptosis, ion conduction, microtubule dynamics, or cytoskeletal organization. Additionally, genes of unknown function are of interest.

### EXAMPLE 3

Confirmation of Differential Expression

Quantitative RT-PCR or similar techniques may be used to confirm the differential expression of mRNA. In cases where commercial antibodies are available, immunoblots (Towbin, Staehlin, & Gordon, 1979) are used to demonstrate differences in protein expression. Protein analysis has the potential to reveal posttranslational modifications that are physiologically relevant.

For quantitative RT-PCR, primers are designed for each candidate gene to be tested. Briefly, sequences of approximately 20 bp with melting temperatures of about 60° C. at 50 mM NaCl and with an adenine or thymidine at the 3′ end are chosen to enhance specificity. The amplicon length is about 100 bp to maximize amplification efficiency, and the selected primers are tested for uniqueness using NCBI Blast searches. Two-step RT-PCR using oligo (dT) or the reverse primer as a gene-specific primer during first-strand cDNA synthesis is performed, optimizing reaction conditions using MasterAmp™ premixes (Invitrogen). The production of a single PCR product of the predicted length, without primer dimers or other undesired products, is confirmed using DNA 500 LabChips™ (Agilent). Real-time RT-PCR (Mx3000P, Stratagene) is then used to quantitate the amount of message in myelinated and demyelinated cultures using SYBR green as the detection method. Melt curves are used to evaluate the specificity of the reactions.

Quantification of the amount of a particular mRNA in the sample is based on a standard curve run at the same time as the samples. Standards are prepared by isolation of PCR amplicons from agarose gels. The DNA concentrations are determined as the average value of multiple dilutions analyzed using DNA 500 LabChips™. A seven-point standard curve consisting of ten-fold serial dilutions of the DNA standard is included on the same qRT-PCR plate as the cDNA samples from myelinated and demyelinated cultures.

A complementary method for determining variations in protein levels in response to demyelination is used for gene products that have commercially-available antibodies. Proteins are extracted by douncing cells in buffer containing protease inhibitors. The Protein and RNA Isolation System (PARIS) from Ambion contains buffers that allow isolation of both protein and RNA from the same cell sample, eliminating uncontrolled variables between cultures. Serial dilutions of whole cell lysates containing equal amounts of protein (as determined by Bradford assays) extracted from myelinated and demyelinated cultures are loaded on SDS-PAGE gels. Immunoblots are performed essentially as described in Towbin, et al., (1979). Commercial antibodies are used to detect the protein of interest and actin or another protein that is not regulated in response to demyelination as determined by the microarray analysis. The internal standard is used for normalization. Detection will be by horseradish peroxidase-conjugated secondary antibodies and chemiluminescence (West Femto SuperSignal™, Pierce). Comparison of the signals from myelinated and demyelinated cultures will be by densitometry (Kodak Image Station).

Experimental Design and Analysis

Analysis of each candidate gene is performed on at least three independent RNA preparations. The microarray results will be used to inform the decision about the appropriate time interval between demyelination and RNA extraction. Triplicate samples from each RNA preparation is analyzed by qRT-PCR and quantified using a standard curve for each gene. For immunoblots, the normalized mean ratio of protein from myelinated and demyelinated cultures from at least three experiments using protein isolated from independently differentiated cultures will be determined.

The method of SYBR green detection for qRT-PCR may be prone to errors if primer dimers or other products are amplified because it measures total double-stranded DNA. RT-PCR reactions are optimized to prevent this problem and melt curves taken after each qRT-PCR is analyzed to indicate problematic reactions. If specificity is a major problem, a second set of primers can be designed. If the problem persists, TaqMan primers can be used.

This example can verify which genes play a significant role in disease progression in MS, as it is expected that at least a two-fold change in expression will occur in each of the independent cultures upon demyelination. The qRT-PCR and immunoblot analysis is used to eliminate false positive results from the microarray experiments and to prioritize the identified genes for further experimentation.

### EXAMPLE 4

This example demonstrates how methods of the present invention can be used to identify genes as being differentially expressed in myelinated and demyelinated cultures

Because the cell culture model contains several cell types including neurons, oligodendrocytes, and astrocytes, the methods of the present invention can include steps to confirm where identified genes are primarily expressed. These steps can be achieved using immunofluorescence microscopy. For example, differentiated cells can be plated on glass cover slips coated with laminin and poly-D-lysine as an attachment substrate and cultured for approximately two weeks to allow myelin formation. Particularly in the case of genes that are upregulated in response to demyelination, cultures can be demyelinated as described above. Cells can then be fixed in cold methanol and incubated with primary and secondary antibodies. Double labeling with an antibody against the protein of interest and antibodies that recognize each of the cell types will be used. Primary antibodies will be selected that have been raised in different species and secondary antibodies will be conjugated to different fluors. For example, to label neurons mouse anti-βIII tubulin (Promega) can be used as the primary antibody assuming that a suitable rabbit primary antibody is available for the protein of interest. Secondary antibodies would then be α-mouse IgG-Rhodamine (Molecular Probes) to label neurons with a red fluor and α-rabbit IgG-FITC (Immunotech) to label the protein of interest with a green fluor. Separate cover slips will be treated in a similar way to label oligodendrocytes or astrocytes and the protein of interest. Antibody dilutions will be optimized to give a good signal using a Zeiss fluorescence microscope with deconvolution capabilities. Rabbit α-galactocerebroside (Chemicon) and mouse α-glial fibrillary acidic protein (GFAP; Chemicon) can be used to identify oligodendrocytes and astrocytes, respectively.

If the use of laminin/poly-D-lysine as an attachment substrate does not allow myelin formation as readily as seen in three-dimensional cultures on Matrigel. Cover slips coated with nanofibers can provide a 3D surface for cell culture without the thickness of Matrigel. These are sold by Donaldson as Ultra-Web™ Synthetic ECM cover slips.

If methanol fixation is not ideal for labeling with all antibodies, formaldehyde fixation is an alternative method. If permeabilization is required for efficient labeling, 0.25% Triton X-100 or saponin can be used. For some molecules such as membrane proteins and lipids (e.g. galactocerebroside), antibody labeling may be more appropriate prior to fixation. Multiple methods may need to be tried in order to find optimal conditions for the proteins of interest.

Some of the genes of interest identified may not have commercially-available antibodies for their gene products. In this case, commercial sources may be used to produce peptide-specific antibodies for those of highest priority. Fluorescent in situ hybridization (FISH) may also be used to label the mRNA. This may provide useful information regarding the cell type with highest expression.

Immunfluorescence microscopy can be used to determine the location and probable site of action within the cell. This information can be particularly useful for establishing function. Cellular locations that can be studied include the nucleus, cytoplasm, mitochondria and plasma membrane. For neurons, additional subcellular features such as synaptic clefts or nodes of Ranvier might be identified. Immunofluorescence microscopy will be performed essentially as above. To differentiate the nucleus from the cytoplasm, Hoechst dye can be used to stain the nucleus blue. After identifying the location as cytoplasmic or nuclear, studies can be designed to further localize the protein of interest to probable organelles. Features such as punctate or diffuse staining as well as general shape and location will inform and direct the appropriate choice of antibodies. Further analysis could involve double labeling to localize proteins to subcompartments within an organelle. The subcellular localization of a protein can be a strong indicator of function and a guide for further functional studies.

Knowing the cellular phenotype of increased or decreased gene expression can help determine the potential of a candidate gene as a good drug target. Genes that have been determined to be upregulated in response to demyelination are to be overexpressed; those that are downregulated are used in gene knockdown studies. General phenotypes are observed as morphological changes by microscopy or loss of cell viability measured by the MTS assay described above. Analysis of other phenotypes may be indicated by the proposed function or cellular localization of a particular gene product.

Transfection efficiencies of neuronal cultures are low. Reagents optimized for this purpose can be used. For example, NeuroPORTER™ Transfection Reagent available from Gene Therapy Systems can be used. This allows plasmid DNA to be transfected directly into neurons and glial cells without the need for viral systems. For overexpression studies, the cDNA of interest can be cloned into a plasmid containing a eukaryotic inducible promoter such as the metallothioneine promoter using standard techniques. Following transfection and induction, effects on cellular phenotypes can be observed as described above.

For gene knockdown studies, siRNA is designed for the genes of interest using web-based tools such as the siRNA Target Finder. Multiple siRNAs to each target are used to show reproducibility of results. Positive and negative controls available from Ambion can monitor transfection efficiency, RNAi induction, and baseline measurements. The ability to rescue any observed phenotypes is investigated by introduction of a plasmid as described for the overexpression studies. Before and after transfection, RNA and protein is extracted from cells using the PARIS system described herein. Protein and mRNA levels of the gene of interest are be measured using qRT-PCR and immunoblots to monitor the extent of gene silencing. Phenotypes are observed as described herein.

Titration of the siRNA to use the lowest effective level can help prevent off target effects. If off target or nonspecific effects are suspected, array analysis can be used to determine the extent and nature of the response. Titration of the siRNA can be used to match as closely as possible the degree of downregulation observed in response to demyelination in order to observe phenotypes that are likely to have physiological significance.

Promising drug targets can be identified based on the examples/experiments described herein. For example, if a gene that is downregulated in response to demyelination and causes a significant decrease in metabolic activity in gene knockdown experiments, therapeutic compounds that upregulate the gene or increase the activity of the gene product would be good candidates for stabilizing the health of the demyelinated axon.

Several references or printed publications, specifically including the references cited below, are cited in this application. All such references or printed publications are expressly incorporated herein by reference in their entirety, and are considered as being part of this disclosure.
Bain, G., Ray, W. J., Yao, M., Gottlieb, D. I. (1996). Retinoic acid promotes neural and represses mesodermal gene expression in mouse embryonic stem cells in culture. Biochem. Biophys. Res. Commun. 223:691-694.
Dennis, M., Spiegler, B. J., Hetherington, C. R. and Greenberg, M. L. (1996). Neuro-psychological sequelae of the treatment of children with medulloblastoma. J. Neurooncol. 29:91-101.
Hutchins, J. B. (1995). Platelet-derived growth factor receptors of mouse central nervous system cells in vitro. J. Comp. Neurol. 360:59-80.
Krauss, R. and Fischbach, G. D. (1998). Cell culture of cholinergic and cholinoceptive neurons from the medial habenula. In: Banker, G. and Goslin, K., eds. Culturing Nerve Cells, 2^{nd} edition. Cambridge, Mass.: MIT Press, pp. 395-418.
Liu, B. P., Fournier, A., GrandPre, T. and Strittmatter, S. M. (2002). Myelin-associated glycoprotein as a functional ligand for the Nogo-66 receptor. Science 297:1190-1193.
Liu, S., Qu, Y., Stewart, T. J., Howard, M. J., Chakrabortty, S., Holekamp, T. F. and McDonald, J. W. (2000). embryonic stem cells differentiate into oligodendrocytes and myelinate in culture and after spinal cord transplantation. Proc. Natl. Acad. Sci. U.S.A. 97:6126-6131.
Mulhern, R. K., Kepner, J. L., Thomas, P. R., Armstrong, F. D., Friedman, H. S. and Kun, L. E. (1998). Neuropsychologic functioning of survivors of childhood medulloblastoma randomized to receive conventional or reduced-dose cranial-spinal irradiation: A Pediatric Oncology group study. J Clin. Oncol. 16:1723-1728.
Mulhern, R. K., Reddick, W. E., Palmer, S. L., Glass, J. O., Elkin, T. D., Kun, L. E., Taylor, J., Langston, J. and Gajjar, A. (1999). Neurocognitive deficits in medulloblastoma survivors and white matter loss. Ann. Neurol. 46:834-841.
Reier, P. J., Tabira, T. and Webster, H. deF. (1978). Hexachlorophene-induced myelin lesions in the amphibian central nervous system. J. Neurol. Sci. 35:257-274.
Schultheiss, T. E., Kun, L. E., Ang, K. K. and Stephens, L. C. (1995). Radiation response of the central nervous system. Int. J. Radiat. Oncol. Biol. Phys. 31:1093-1112.
Stewart, E. S. and Cohen, D. G. (1998). Central nervous system tumors in children. Semin. Oncol. Nurs. 14:34-42.
Towbin, H., Staehlin, T. and Gordon, J. (1979). Electrophoretic transfer of proteins from polyacrylamide gels to nitrocellulose sheets: Procedure and some applications. Proc. Natl. Acad. Sci. U.S.A. 76:4350-4354.
Virta, A., Patronas, N., Raman, R., Dwyer, A., Barnett, A., Bonavita, S., Tedeschi, G. and Lundbom, N. (2000). Spectroscopic imaging of radiation-induced effects in the white matter of glioma patients. Magn. Reson. Imaging 18:851-857.
Vrdoljak, E., Bill, C. A., Stephens, L. C., Van der Kogel, A. J., Ang, K. K. and Tofilon, P. J. (1992). Radiation-induced apoptosis of oligodendrocytes in vitro. Int. J. Radiat. Biol. 62:475-480.
Waxman, S. G., Kocsis, J. D. and Black, J. A. (1995). Pathophysiology of demyelinated axons. In: Waxman, S. G., Kociss, J. D. and Stys P. K., eds. The Axon: Structure, Function, and Pathophysiology. New York, N.Y.: Oxford University Press, pp. 438-461.
Williams, R. L., Hilton, D. J., Pease, S., Willson, T. A., Stewart, C. L., Gearing, D. P., Wagner, E. F., Metcalf, D., Nicola, N. A. and Gough, N. M. (1988). Myeloid leukaemia inhibitory factor maintains the developmental potential of embryonic stem cells. Nature 336:684-687.
Zhu, B., Moore, G. R. W., Zwimpfer, T. J., Kastrukoff, L. F., Dyer, J. K., Steeves, J. D., Paty, D. W. and Cynader, M. S. (1999). Axonal cytoskeleton changes in experimental optic neuritis. Brain Res. 824:204-217.
Arredondo, L. R., Lovett-Racke, A. E., & Racke, M. K. (2003). Experimental autoimmune encephalomyelitis. In R. M. Herndon (Ed.), Multiple sclerosis: Immunology, pathology, and pathophysiology (pp. 79-103). New York, N.Y.: Demos.
Carmody, R. J., Hilliard, B., Maguschak, K., Chodosh, L. A., & Chen, Y. H. (2002). Genomic scale profiling of autoimmune inflammation in the central nervous system: The nervous response to inflammation. Journal of Neuroimmunology, 133, 95-107.
Dalakas, M. C. (1995). Pathogenetic mechanisms of post-polio syndrome: Morphological, electrophysiological, virological, and immunological correlations. Annals of the New York Academy of Sciences, 753, 167-185.
Hemdon, R. M. (2002). Medical hypothesis: Why secondary progressive multiple sclerosis is a relentlessly progressive illness. Archives of Neurology, 59, 301-304.
Ibrahim, S. M., Mix, E., Bottcher, T., Koczan, D., Gold, R., Rolfs, A., et al. (2001). Gene expression profiling of the nervous system in murine experimental autoimmune encephalomyelitis. Brain, 124, 1927-1938.
Lock, C., Hermans, G., Pedotti, R., Brendolan, A., Schadt, E, Garren, H., et al. (2002). Gene-microarray analysis of multiple sclerosis lesions yields new targets validated in autoimmune encephalomyelitis. Nature Medicine, 8, 500-508.
Maas, K., Chan, S., Parker, J., Slater, A., Moore, J., Olsen, N., et al. (2002). Cutting edge: Molecular portrait of human autoimmune disease. The Journal of Immunology, 169, 5-9.
McQuarrie, I., Brady, S. T., & Lasek, R. J. (1986). Diversity in the axonal transport of structural proteins: Major differences between optic and spinal axons in the rat. Journal of Neuroscience, 6, 1593-1605.
Mix, E., Pahnke, J., & Ibrahim, S. M. (2002). Gene-expression profiling of experimental autoimmune encephalomyelitis. Neurochemical Research, 27, 1157-1163.
Mycko, M. P., Papoian, R., Boschert, U., Raine, C. S., & Selmaj, K. W. (2003). cDNA microarray analysis in multiple sclerosis lesions: Detection of genes associated with disease activity. Brain, 126, 1048-1057.
Nicot, A., Ratnakar, P. V., Ron, Y., Chen, C.-C., & Elkabes, S. (2003). Regulation of gene expression in experimental autoimmune encephalomyelitis indicates early neuronal dysfunction. Brain, 126, 398-412.
Paschen, W., & Frandsen, A. (2001). Endoplasmic reticulum dysfunction—a common denominator for cell injury in acute and degenerative diseases of the brain? Journal of Neurochemistry, 79, 719-725.
Ramanathan, M., Weinstock-Guttman, B., Nguyen, L. T., Badgett, D., Miller, C., Patrick, K., et al. (2001). In vivo gene expression revealed by cDNA arrays: The pattern in relapsing-remitting multiple sclerosis patients compared with normal subjects. Journal of Neuroimmunology, 116, 213-219.
Spira, M. E., Oren, R., Dormann, A., Ilouz, N., & Lev, S. (2001). Calcium, protease activation, and cytoskeleton remodeling underlie growth cone formation and neuronal regeneration. Cellular and Molecular Neurobiology, 21, 591-604.
Towbin, H., Staehlin, T., & Gordon, J. (1979). Electrophoretic transfer of proteins from polyacrylamide gels to nitrocellulose sheets: Procedure and some applications. Proceedings of the National Academy of Sciences, U.S.A., 76, 4350-4354.
Whitney, L. W., Ludwin, S. K., McFarland, H. F., & Biddison, W. E. (2001). Microarray analysis of gene expression in multiple sclerosis and EAE identifies 5-lipoxygenase as a component of inflammatory lesions. Journal of Neuroimmunology, 121, 40-88.
Williams, R. L., Hilton, D. J., Pease, S., Willson, T. A., Stewart, C. L., Gearing, D. P., et al. (1988). Myeloid leukaemia inhibitory factor maintains the developmental potential of embryonic stem cells. Nature, 336, 684-687.
Yang, Y. H., & Speed, T. (2003). Design of microarray expression experiments. In D. Bowtell & J. Sambrook (Eds.), DNA Microarrays: A molecular cloning manual (pp. 513-525). Cold Spring Harbor, N.Y.: Cold Spring Harbor Laboratory Press.
Zhu, B., Moore, G. R. W., Zwimpfer, T. J., Kastrukoff, L. F., Dyer, J. K., Steeves, J. D., et al. (1999). Axonal cytoskeleton changes in experimental optic neuritis. Brain Research, 824, 204-217.

The invention being described, it will be apparent to those skilled in the art that various modifications and variations can be made in the present invention without departing from the scope or spirit of the invention. Other embodiments of the invention will be apparent to those skilled in the art from consideration of the specification and practice of the invention disclosed herein.

This application claims benefit to U.S. application No. 60/624,738, filed Nov. 2, 2004, and U.S. application Ser. No. 11/265,587, filed Nov. 2, 2005, the contents of which are incorporated herein by reference.

1-21. (canceled)

22. A method of creating an in vitro demyelination model system for demyelination study, comprising:
providing embryonic stem cells;
culturing said stem cells to form myelin;
combining said cultured stem cells with a solution that includes at least one of hexachlorophene and/or lysophosphatidylcholine to cause demyelination of said stem cells and form demyelinated stem cells;
introducing a compound suspected of promoting remyelination to said demyelinated stem cells;
monitoring the stem cells for signs of remyelination,

23. The method of claim 22, wherein the combination step includes contacting said stem cells with a solution that comprises about 10 μg/ml hexachlorophene.

24. The method of claim 22, wherein the combination step comprises contacting the stem cells and the solution for about 10 minutes.

25. The method of claim 22, further comprising the step of identifying genes that are up-regulated or down-regulated in response to demyelination of CNS axons.

26. The method of claim 22, further considering the up-regulation and down-regulation of a gene to determine a therapeutic agent to address said up-regulation or down-regulation to treat demyelination and/or enhance remyelination.

27. The method of claim 22, further comprising the step of evaluating the combination evaluation of gene expression profiles of demyelination by microarrays.

28. In combination:
(a) embryonic stem cells that have been differentiated and cultured to form myelin; and
(b) a solution that includes at least one of hexachlorophene and/or lysophosphatidylcholine to cause demyelination of said differentiated stem cells and form demyelinated axons;
the combination producing cells that receive a compound suspected of promoting remyelination to said demyelinated cells; and
the combination producing cells capable of being monitored to determine remyelination of differentiated stem cells.

29. The combination of claim 28, wherein the solution comprises about 10 μg/ml hexachlorophene.

30. The combination of claim 28, wherein the (a) and (b) are in contact with one another for about 10 minutes.

31. The combination of claim 28, wherein the combination allows for evaluation of gene expression profiles of demyelination by microarrays.

32. An in vitro demyelination model system for demyelination study produced by:
providing embryonic stem cells that have been differentiated and cultured to form myelin;
combining said cultured stem cells with a solution that includes at least one of hexachlorophene and/or lysophosphatidylcholine to cause demyelination of said differentiated stem cells and form demyelinated cells; and
introducing a compound suspected of promoting remyelination to said demyelinated cells; wherein
the model is adaptable to monitor cells for signs of remyelination.

33. The model of claim 32, wherein the solution comprises about 10 μg/ml hexachlorophene.

34. The model of claim 32, wherein the differentiated stem cells and the solution are combined for about 10 minutes.

35. The model of claim 32, being adaptable to identify genes that are up-regulated or down-regulated in response to demyelination of CNS axons.

36. The method of claim 32, being adaptable for up-regulation and down-regulation of a gene to determine a therapeutic agent to address said up-regulation or down-regulation to treat demyelination and/or enhance remyelination.

37. The model of claim 32, being adaptable to allow for evaluation of gene expression profiles of demyelination by microarrays.
